(19) United States
(12) Patent Application Publication
(10) Pub. No.: US 2007/0270719 A1
(43) Pub. Date: Nov. 22, 2007

(54) Automatic speed setting system for bicycle use

(76) Inventor: Star Jwo, Miaoli Hsien (TW)

Correspondence Address: HARNESS, DICKEY & PIERCE, P.L.C., P.O. BOX 8910, RESTON, VA, 20195, US

(21) Appl. No.: 11/416,179

(22) Filed: May 3, 2006

(51) Int. Cl. A61B 5/103

(52) U.S. Cl. 600/587

(57) ABSTRACT

An automatic speed setting system is adapted for use with a bicycle that includes a gearing mechanism having gear ratios. The automatic speed setting system includes a physiological parameter detecting unit adapted for detecting a physiological parameter of a cyclist riding the bicycle, a physiological parameter processing unit coupled to the physiological parameter detecting unit and operable so as to calculate physiological parameter data of the cyclist with reference to the physiological parameter, a signal generating unit coupled to the physiological parameter processing unit and operable so as to generate a drive signal with reference to the physiological parameter data, and an automatic shifting unit coupled to the signal generating unit and adapted to operate the gearing mechanism of the bicycle in a selected one of the gear ratios in response to the drive signal.

### BACKGROUND OF THE INVENTION

1. Field of the Invention

The present invention relates to an automatic speed setting system for a bicycle. More particularly, the present invention relates to an automatic speed setting system for a bicycle that automatically varies a gear ratio of a gearing mechanism of the bicycle in accordance with changes in a physiological parameter of a cyclist riding the bicycle.

2. Description of the Related Art

Conventional automatic speed setting systems for bicycles have been disclosed, in which a gear ratio of a gearing mechanism of the bicycle is varied automatically. That is, the gear ratio of the gearing mechanism is automatically changed on the basis of riding speed. This is typically realized by assigning a predetermined riding speed range to each gear ratio of the gearing mechanism, and changing the gear ratio when the present riding speed corresponds to a different gear ratio than the current gear ratio.

However, a drawback of the conventional system is that the physical state of the cyclist is not a consideration when controlling the gearing mechanism. As a result, shifting may be effected into a higher gear ratio sooner than the cyclist is physically prepared to handle the extra effort associated with the higher speed, or into a lower gear ratio earlier than physically required by the cyclist.

### SUMMARY OF THE INVENTION

Therefore, the object of the present invention is to provide an automatic speed setting system for a bicycle that automatically varies a gear ratio of a gearing mechanism of the bicycle in accordance with changes in a physiological parameter of a cyclist riding the bicycle.

The automatic speed setting system of this invention is adapted for use with a bicycle that includes a gearing mechanism having a plurality of gear ratios. The automatic speed setting system comprises: a physiological parameter detecting unit adapted for detecting a physiological parameter of a cyclist riding the bicycle; a physiological parameter processing unit coupled to the physiological parameter detecting unit and operable so as to calculate physiological parameter data of the cyclist riding the bicycle with reference to the physiological parameter obtained by the physiological parameter detecting unit; a signal generating unit coupled to the physiological parameter processing unit and operable so as to generate a drive signal with reference to the physiological parameter data calculated by the physiological parameter processing unit; and an automatic shifting unit coupled to the signal generating unit and adapted to operate the gearing mechanism of the bicycle in a selected one of the gear ratios in response to the drive signal received from the signal generating unit.

### BRIEF DESCRIPTION OF THE DRAWINGS

Other features and advantages of the present invention will become apparent in the following detailed description of the preferred embodiment with reference to the accompanying drawings, of which:

FIG. 1 is a schematic block diagram of an automatic speed setting system for a bicycle according to a preferred embodiment of the present invention; and

FIG. 2 is a fragmentary perspective view of the preferred embodiment in a state mounted to a bicycle.

### DETAILED DESCRIPTION OF THE PREFERRED EMBODIMENT

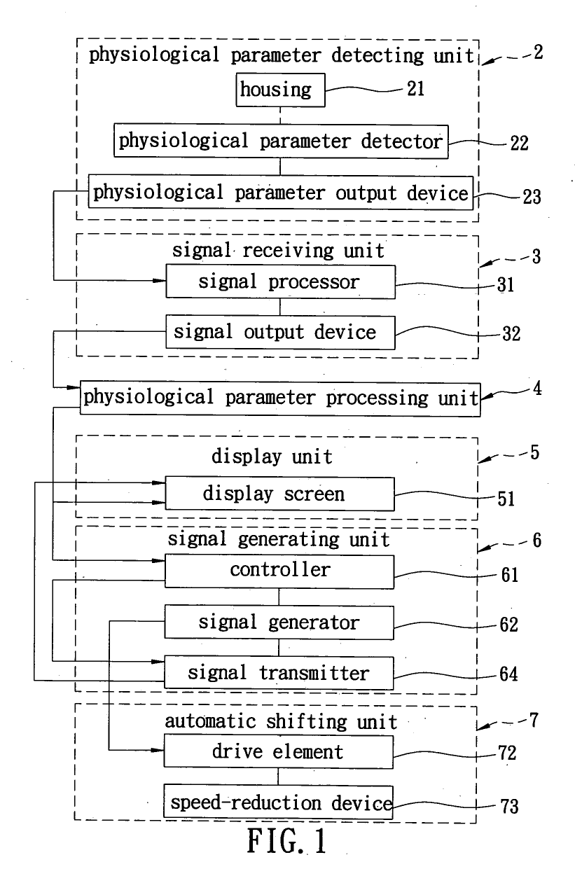

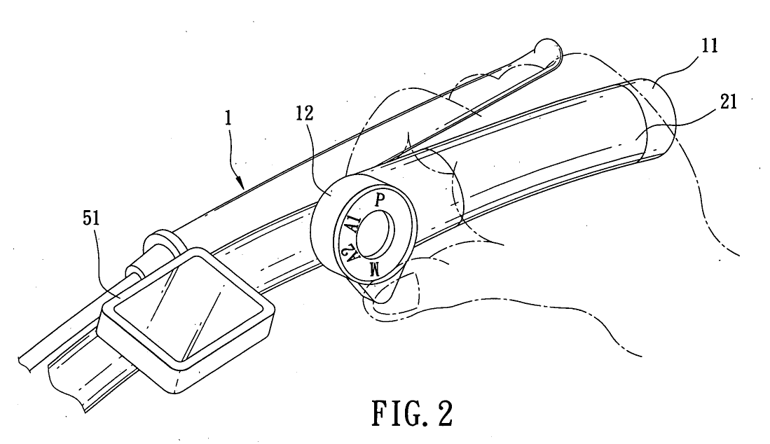

Referring to FIGS. 1 and 2, an automatic speed setting system according to a preferred embodiment of the present invention is adapted for use with a bicycle 1. The bicycle 1 includes handlebars 11 (only one is shown), and a conventional gearing mechanism (not shown) having a plurality of gear ratios. In this embodiment, the automatic speed setting system includes a physiological parameter detecting unit 2, a physiological parameter processing unit 4, a display unit 5, a signal generating unit 6, and an automatic shifting unit 7. In one embodiment, the automatic speed setting system further includes a shifter 12 mounted on the handlebars 11 of the bicycle 1, and that is operated by a cyclist riding the bicycle 1 to turn the automatic speed setting system ON and OFF, and to control the automatic speed setting system into different auto-shifting modes. For the description that follows, it is assumed that the shifter 12 has been controlled by the cyclist to turn the automatic speed setting system ON, and to control the automatic speed setting system into one of the auto-shifting modes.

The physiological parameter detecting unit 2 is adapted for detecting a physiological parameter of a cyclist riding the bicycle 1. In one embodiment, the physiological parameter detecting unit 2 is adapted to detect at least one of a heart rate and a pulse rate of the cyclist riding the bicycle 1. The physiological parameter detecting unit 2 includes a housing 21 adapted to be placed in contact with the body of the cyclist riding the bicycle 1, a physiological parameter detector 22 mounted to the housing 21 and adapted to detect the physiological parameter of the cyclist, and a physiological parameter output device 23 coupled to the physiological parameter detector 22 and the physiological parameter processing unit 4 and providing the physiological parameter of the cyclist to the physiological parameter processing unit 4.

In one embodiment, the automatic speed setting system further includes a signal receiving unit 3 coupled to the physiological parameter output device 23 and the physiological parameter processing unit 4. In this case, there is no direct coupling between the physiological parameter output device 23 and the physiological parameter processing unit 4, and the signal receiving unit 3 may relay the physiological parameter from the physiological parameter detecting unit 2 to the physiological parameter processing unit 4. The signal receiving unit 3 is adapted to receive from the bicycle 1 an auxiliary signal associated with any one of a road slope, a current gear ratio of the gearing mechanism, pedaling torque, and riding speed. The signal receiving unit 3 includes a signal processor 31 to receive the physiological parameter and the auxiliary signal, and a signal output device 32 to relay the physiological parameter and the auxiliary signal to the physiological parameter processing unit 4.

The physiological parameter processing unit 4 is operable so as to calculate physiological parameter data of the cyclist riding the bicycle 1 with reference to the physiological parameter obtained by the physiological parameter detecting unit 2. In one embodiment, the physiological parameter data is calculated by the physiological parameter processing unit 4 with reference to both the physiological parameter and the auxiliary signal. The physiological parameter data calculated by the physiological parameter processing unit 4 includes at least one of a physiological parameter present state analysis, physiological parameter range analysis, physiological parameter variability and oxygen consumption analysis, and physiological parameter variability relative to time analysis. An example of physiological parameter range analysis is that of calculating a maximum heart rate of the cyclist riding the bicycle 1 as:
maximum heart rate=220−age.

The display unit 5 is coupled to the physiological parameter processing unit 4 and the signal generating unit 6, and includes a display screen 51. The display unit 5 is operable so as to display data on the display screen 51 associated with at least one of the physiological parameter and the auxiliary signal. That is, through control by the physiological parameter processing unit 4 or using a signal transmitted by the signal generating unit 6 (to be described below), the display unit 5 displays the data in the form of at least one of a heart rate of the cyclist riding the bicycle 1, an electrocardiogram of the cyclist riding the bicycle 1, present riding speed, distance traveled by the bicycle 1, elapsed riding time, present pedaling torque, and current gear ratio of the gearing mechanism of the bicycle 1.

The signal generating unit 6 is coupled to the physiological parameter processing unit 4, and is operable so as to generate a drive signal with reference to the physiological parameter data calculated by the physiological parameter processing unit 4. The signal generating unit 6 includes a controller 61 coupled to the physiological parameter processing unit 4, a signal generator 62 coupled to the controller 61 and the automatic shifting unit 7, and a signal transmitter 64 coupled to the controller 61. The controller 61 controls the signal generator 62 to generate the drive signal and to provide the drive signal to the automatic shifting unit 7. The controller 61 controls the signal transmitter 64 to transmit a display signal associated with the drive signal to the display unit 5.

The automatic shifting unit 7 is coupled to the signal generating unit 6, and is adapted to operate the gearing mechanism of the bicycle 1 in a selected one of the gear ratios in response to the drive signal received from the signal generating unit 6. The automatic shifting unit 7 includes a drive element 72 adapted to operate the gearing mechanism in the selected one of the gear ratios, and a speed-reduction device 73 adapted to couple the drive element 72 to the gearing mechanism of the bicycle 1. In one embodiment, the drive element 72 is a motor.

With the use of the above-described automatic speed setting system of the present invention, the gear ratio of the gearing mechanism may be controlled to best correspond to the physical state of the cyclist riding the bicycle 1. For example, if the physiological parameter detecting unit 2 is adapted for detecting the heart rate of the cyclist, the gear ratio of the gearing mechanism may be controlled such that shifting is effected into a higher gear ratio when the heart rate of the cyclist is sufficiently low, and into a lower gear ratio when the heart rate of the cyclist reaches a certain threshold value.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. An automatic speed setting system adapted for use with a bicycle that includes a gearing mechanism having a plurality of gear ratios, said automatic speed setting system comprising:
a physiological parameter detecting unit adapted for detecting a physiological parameter of a cyclist riding the bicycle;
a physiological parameter processing unit coupled to said physiological parameter detecting unit and operable so as to calculate physiological parameter data of the cyclist riding the bicycle with reference to the physiological parameter obtained by said physiological parameter detecting unit;
a signal generating unit coupled to said physiological parameter processing unit and operable so as to generate a drive signal with reference to the physiological parameter data calculated by said physiological parameter processing unit; and
an automatic shifting unit coupled to said signal generating unit and adapted to operate the gearing mechanism of the bicycle in a selected one of the gear ratios in response to the drive signal received from said signal generating unit.

2. The automatic speed setting system of claim 1, wherein said physiological parameter detecting unit includes a housing adapted to be placed in contact with the body of the cyclist riding the bicycle, a physiological parameter detector mounted to said housing and adapted to detect the physiological parameter of the cyclist, and a physiological parameter output device coupled to said physiological parameter detector and said physiological parameter processing unit and providing the physiological parameter of the cyclist to said physiological parameter processing unit.

3. The automatic speed setting system of claim 1, wherein said signal generating unit includes a signal generator coupled to said automatic shifting unit, and a controller coupled to said signal generator and said physiological parameter processing unit and controlling said signal generator to generate the drive signal and to provide the drive signal to said automatic shifting unit.

4. The automatic speed setting system of claim 1, further comprising a signal receiving unit coupled to said physiological parameter detecting unit and said physiological parameter processing unit, said signal receiving unit relaying the physiological parameter from said physiological parameter detecting unit to said physiological parameter processing unit, and being adapted to receive from the bicycle an auxiliary signal associated with any one of a road slope, a current gear ratio of the gearing mechanism, pedaling torque, and riding speed.

5. The automatic speed setting system of claim 4, wherein the physiological parameter data is calculated by said physiological parameter processing unit with reference to the physiological parameter and the auxiliary signal, the physiological parameter data including at least one of a physiological parameter present state analysis, physiological parameter range analysis, physiological parameter variability and oxygen consumption analysis, and physiological parameter variability relative to time analysis.

6. The automatic speed setting system of claim 4, wherein said signal receiving unit includes a signal processor to receive the physiological parameter and the auxiliary signal, and a signal output device to relay the physiological parameter and the auxiliary signal to said physiological parameter processing unit.

7. The automatic speed setting system of claim 4, further comprising a display unit coupled to said physiological parameter processing unit and said signal generating unit, said display unit being operable so as to display data associated with at least one of the physiological parameter and the auxiliary signal.

8. The automatic speed setting system of claim 7, wherein said display unit displays the data in the form of at least one of a heart rate of the cyclist riding the bicycle, an electrocardiogram of the cyclist riding the bicycle, present riding speed, distance traveled by the bicycle, elapsed riding time, present pedaling torque, and current gear ratio of the gearing mechanism of the bicycle.

9. The automatic speed setting system of claim 1, further comprising a display unit coupled to said physiological parameter processing unit and operable so as to display the physiological parameter data.

10. The automatic speed setting system of claim 1, wherein said automatic shifting unit includes a drive element adapted to operate the gearing mechanism in the selected one of the gear ratios, and a speed-reduction device adapted to couple said drive element to the gearing mechanism of the bicycle.

11. The automatic speed setting system of claim 10, wherein said drive element is a motor.

12. The automatic speed setting system of claim 1, wherein said physiological parameter detecting unit is adapted to detect at least one of a heart rate and a pulse rate of the cyclist riding the bicycle.
